(19) United States
(12) Patent Application Publication
(10) Pub. No.: US 2003/0110538 A1
(43) Pub. Date: Jun. 12, 2003

(54) Novel 'oxy' CMS brassica napus corrrected for chlorosis using hexaploid bridging material generated through protoplast fusion and a method of producing the plant

(76) Inventors: Deepak Pental, New Delhi (IN); Arundhati Mukhopadhyay, New Delhi (IN); Vibha Gupta, New Delhi (IN); Neelakantan Arumugam, New Delhi (IN); Yashpal Singh Sodhi, New Delhi (IN); Jagdish Kumar Verma, New Delhi (IN); Akshay Kumar Pradhan, New Delhi (IN)

Correspondence Address: BAKER & BOTTS, 30 ROCKEFELLER PLAZA, NEW YORK, NY, 10112

(21) Appl. No.: 10/206,642

(22) Filed: Jul. 26, 2002

Related U.S. Application Data
(63) Continuation of application No. PCT/IN00/00006, filed on Jan. 28, 2000.

(51) Int. Cl. A01H 5/00

(52) U.S. Cl. 800/306; 800/282

(57) ABSTRACT

The present invention relates to a novel cytoplasmic male sterile (CMS) Brassica napus plant containing improved ‘oxy’ cytoplasm i.e. chloroplast from Brassica oleracea and recombinant mitochondria from Brassica oxyrrhina, a process for developing the said ‘oxy’ CMS Brassica napus plants by using hexaploid ‘oxy’ CMS Brassica plants, obtained through somatic hybridization, as the bridging material and backcrossing these for at least five generations to transfer the novel ‘oxy’ CMS character to Brassica napus plants and a method for producing the improved hexaploid male sterile ‘oxy’ CMS Brassica plants mediated through protoplast fusion and regeneration of the somatic hybrids and to the transfer of this improved ‘oxy’ cytoplasm to tetraploid Brassica napus (AACC) plants through conventional backcross breeding for five generations.

### FIELD

The present invention relates to cytoplasmic male sterile Brassica napus plants containing improved ‘oxy’ cytoplasm, i.e. recombined ‘oxy’ CMS mitochondria and chloroplast of cultivated Brassica species. The invention also relates to the methods for obtaining the said male sterile plants by using hexaploid male sterile Brassica plants, generated through somatic cell hybridization, as bridging materials to transfer this ‘oxy’ CMS character to the tetraploid Brassica napus plants through conventional back-cross breeding.

### BACKGROUND

Brassica crops are known to be source of vegetable oil, food and condiment. In recent years demand for the aforesaid products from Brassica plants has increased sharply necessitating increase in the productivity. Cytoplasmic male sterility (CMS) system is one of the most widely used means for the development of hybrid varieties and increasing productivity. CMS is a maternally inherited phenomenon, which manifests itself as the inability to produce functionally viable pollen grains, the male reproductive unit in plants. Genetic determinants of the male sterility are carried by the cytoplasmic organelle, the mitochondria. At each sexually produced generation, the CMS character is transmitted from the mother to the progeny.

A number of CMS systems are known in crop Brassica, most of which are alloplasmic in nature. Alloplasmic CMS plants contain cytoplasm (mitochondria and chloroplast) of an alien species in the nuclear background of a cultivated Brassica plant. One such CMS system called ‘oxy’ was developed through the transfer of Brassica oxyrrhina cytoplasm in the nuclear background of cultivated Brassica plant through conventional back-cross breeding (Prakash and Chopra 1990, TAG 79: 285-87). These plants were completely and stably male sterile but did not possess satisfactory agronomic characteristics. The major problem associated with these ‘oxy’ CMS plants was chlorosis—lack of greenness of plants, resulting in poor yield and thus rendering the CMS unsuitable for commercial use. Chlorosis is the manifestation of the incompatibility between the alien chloroplast and the nuclear genome in the alloplasmic CMS plants Correction of chlorosis through exchange of chloroplast by protoplast fusion has been reported for ‘ogu’ CMS lines of both spring (Pelletier et al 1983, Mol. Gen. Genet 191: 244-250; Menczel et al 1987, Plant cell Rep. 6: 98-101) and winter varieties of rape (Jarl and Bornman 1988, Hereditas 108: 97-102) and for ‘tour’ CMS in B. napus (Stiewe and Robellen 1994, Plant Breeding 113: 294-304; Arumugam et al 1996, TAG 92: 760-768)

The ‘oxy’ CMS Brassica plants as described by Prakash and Chopra (Supra) are chlorotic and not suitable for commercial use. Correction of chlorosis for ‘oxy’ CMS in Brassica juncea was earlier reported by Kirti et al (1993, Genet. Res. Cambridge 62: 11-14), wherein they described the development of a solitary chlorosis-corrected male-sterile ‘oxy’ CMS B. juncea, that was obtained through a rare event of chloroplast recombination mediated through protoplast fusion. Kirti et al. (supra) fused protoplasts derived from ‘oxy’ CMS B. juncea and normal B. juncea to produce 123 allo-octaploid B. juncea. These allo-octaploid B. juncea upon three subsequent back-crossings with normal B. juncea, produced only one normal green male sterile allotetraploid ‘oxy’ B. juncea, having B. oxyrrhina mitochondria and recombined chloroplast of B. juncea and B. oxyrrhina.

Perusal of preceding instances emphasizes the importance of the technique of protoplast fusion/somatic cell hybridization to rectify the problem of chlorosis associated with CMS systems. In other words, corrective measure to remove chlorosis can be effected only through the process of somatic cell hybridization, wherein protoplasts from CMS plant cells containing alien mitochondria and chloroplasts and protoplasts from normal plant cells containing cultivated mitochondria and chloroplasts, are fused together to bring all the organelles under a common cytoplasmic milieu These organelles in the resultant hybrid cells interact with each other: while the mitochondria recombine, the chloroplasts segregate. From the resultant hybrid cells those containing the desirable chloroplast and CMS mitochondria are selected for further development This development is facilitated by a unique property, cellular totipotency, possessed by the plant cells whereby a single cell can regenerate into an entire plant. The above mentioned hybrid cells with desirable chloroplast and CMS mitochondria can thus be regenerated to produce CMS plants corrected for chlorosis.

To solve the problem of ‘oxy’ CMS related chlorosis in B. napus, the applicants set out to devise a suitable fusion protocol for removing the undesirable chloroplasts of ‘oxy’ CMS by substituting these with chloroplasts of ‘oleracea origin and for simultaneously retaining the male sterility character. The normal cultivated B. napus plants contain chloroplasts of B. oleracea, the natural female progenitor. Thus the B. juncea material developed by Kirti et al (supra) can not be used directly to develop chlorosis-corrected ‘oxy’ CMS B. napus since it does not contain ‘oleracea’ chloroplast.

### OBJECTS

The main object of the invention is to develop cytoplasmic male sterile Brassica napus plants (AACC) which contain improved ‘oxy’ CMS cytoplasm i.e. recombined ‘oxy’ CMS mitochondria and chloroplast of Brassica oleracea.

Yet another object is to develop hexaploid cytoplasmic male sterile Brassica plants (AABBCC) containing recombined ‘oxy’ mitochondria and chloroplast from B. oleracea, through somatic cell hybridization, as the bridging material for transferring the improved ‘oxy’ CMS cytoplasm to tetraploid Brassica napus (AACC) plants.

A further object is to back-cross the male sterile hexaploid Brassica plants for at least five generations to transfer cytoplasm mediating the improved ‘oxy’ CMS characters to cultivated Brassica napus

### SUMMARY

Accordingly, the invention provides novel cytoplasmic male sterile Brassica napus plants corrected for chlorosis and containing improved ‘oxy’ CMS character. The said CMS plants can be developed by using hexaploid ‘oxy’ CMS Brassica plants, obtained through somatic hybridization, as the bridging material and back-crossing these for at least five generations to transfer the novel ‘oxy’ CMS character to Brassica napus plants. Further, the invention provides a method for producing the improved hexaploid male sterile ‘oxy’ CMS Brassica plants, the said method comprising the steps of.

a) fusing protoplasts of ‘oxy’ CMS Brassica juncea (AABB) with normal Brassica oleracea (CC) to produce hexaploid somatic hybrid plants (AABBCC),

b) screening of the somatic hybrids for identifying the desirable hexaploid plants containing recombined CMS mitochondria from ‘oxy’ cytoplasm and the chloroplast of B. oleracea for using in the back-crossing programme.

### DETAILED DESCRIPTION

The invention relates to stable ‘oxy’ CMS Brassica napus plants corrected for chlorosis These plants contain recombined ‘oxy’ CMS mitochondria and chloroplast from B. oleracea. The plants of the invention were produced by back-crossing with hexaploid cytoplasmic male sterile Brassica plants containing improved ‘oxy’ CMS characters, i.e. recombined ‘oxy’ CMS mitochondria and chloroplast from B. oleracea. These hexaploid cytoplasmic male sterile Brassica plants were produced by somatic cell hybridization followed by selection of the desired types of hybrids using molecular methods. The protoplasts of B. juncea (AABB) containing ‘oxy’ CMS cytoplasm were fused with protoplasts of normal B. oleracea (CC) following High pH/Ca⁺⁺ method. The hybrids cells were regenerated into plantlets which were screened for the presence of chloroplast from B. oleracea and recombined ‘oxy’ CMS mitochondria. The selected hybrids were back-crossed with B. napus for at least five generations in order to transfer the desirable CMS cytoplasm

Accordingly, the invention provides stable chlorosis-corrected, improved ‘oxy’ CMS Brassica napus (AACC)plants and parts or seeds thereof, whose cytoplasm is provided via protoplast fusion and contains chloroplasts from B. oleracea (CC) and either recombined or native mitochondria from B. oxyrrhina. The said improved ‘oxy’ CMS containing Brassica napus (AACC) plants have the following characteristics:

a) height of 156 cm to 185 cm, average height of about 170 cm,

b) 4 to 6 primary and 2 to 4 secondary branches, averaging 5 and 3 respectively,

c) main shoot having length of about 85 cm,

d) initiation of flowering within about 90 days,

e) having 59 to 78 pods on the main shoot, average of 69,

f) pod density of about 0.81 to 1.09 cm on the main shoot,

g) having about 22 seeds/pods, and

h) oblong to lanceolate leaves with slightly serrated or entire margins and smooth, non-hirsute surface.

Further the invention provides a process for the development of said stable chlorosis-corrected, improved ‘oxy’ CMS Brassica napus plants, said process comprising the steps of:

i) developing hexaploid cytoplasmic male sterile (CMS) Brassica plants (AABBCC) containing improved ‘oxy’ cytoplasm i.e. chloroplast from Brassica oleracea (CC) and either recombinant or unmodified male sterile mitochondria from Brassica oxyrrhina through the process of somatic cell hybridization,

ii) back-crossing the selected hexaploid male sterile somatic hybrids to B. napus to produce F1 generation,

iii) subjecting the F1 generation to further back-crossings, at least up to BC5 generation to produce stable male sterile lines with improved ‘oxy’ CMS in B. napus, and

iv) verification of the cytoplasmic characteristics of the BC5 B. napus plants by molecular analysis to establish the faithful transmission of the improved cytoplasmic characteristics from the hexaploid somatic hybrids through the five backcross generations.

In an embodiment, the Brassica napus used for back-crossing in steps (ii) and (iii) is Brassica napus var ISN 706 (Indian Synthetic napus 706).

Further, the invention provides hexaploid cytoplasmic male sterile ‘oxy’ CMS Brassica plants containing improved ‘oxy’ cytoplasm i.e. chloroplast from Brassica oleracea and either recombinant or unmodified male sterile mitochondria from Brassica oxyrrhina, said hexaploid CMS plants obtained through somatic cell hybridisation.

In an embodiment, the invention provides a method for the development of hexaploid male sterile CMS Brassica plants wherein the said plant has the following characteristics:

i) height of about 140-190 cm, averaging 165 cm,

ii) oblong to lanceolate leaves with serrated or entire margins and smooth, non-hirsute surface,

iii) 3-6 primary branches, and 6-10 secondary branches bearing terminal and/or axillary, indeterminate racemose inflorescence,

iv) initiation of flowering within 60-70 days,

v) pod length of about 4-5 cm, and

vi) having about 4-6 seeds per pod.

In yet another embodiment, the invention provides a method for the development of hexaploid cytoplasmic male sterile ‘oxy’ CMS Brassica plants containing improved ‘oxy’ cytoplasm, said method comprising the steps of:

(i) isolation of protoplasts from ‘oxy’ CMS B. juncea plants (AABB) and B. oleracea (cc) plants,

(ii) separation of protoplasts from the cell debris by conventional methods,

(iii) fusion of parental protoplasts using conventional methods,

(iv) cultivation of microcolonies, in a suitable growth media,

(v) selection of hybrid (AABBCC) colonies from the milieu of microcolonies derived from various non-fused parental cells, using appropriate selection agents or markers,

(vi) inducing further growth of the hybrid colonies to form calli and differentiation of shoots from the selected colonies employing suitable media,

(vii) inducing development of roots from the shoots employing a suitable medium,

(viii) transferring the rooted plantlets to soil to obtain ‘oxy’ CMS hexaploid (AABBCC) Brassica somatic hybrid plants,

(ix) screening of the hybrid plants for pollen fertility on the basis of morphological observations,

(x) confirming the hybrid nature of the plants by molecular analysis of their nuclear genomes, and,

(xi) analyzing the organelle compositions for the presence of ‘oleracea’ type chloroplast and recombined ‘oxy’ mitochondrial genome which is indicative of the improved ‘oxy’ CMS systems corrected for chlorosis.

In yet another embodiment, the progenitors of hexaploid ‘oxy’ CMS Brassica plants are selected from Brassica juncea (AABB) and B. oleracea (CC) plants. In a further embodiment, the protoplasts are isolated from Brassica juncea and B. oleracea plants.

In another embodiment, the protoplasts are isolated from plant parts of B. juncea and B. Oleracea selected from finely chopped tissues such as leaves, internodes, and petioles.

In still another embodiment, the tissues of the plant parts are treated with cell wall digesting enzymes selected from cellulase and macerozyme and kept overnight in dark or low light intensity at 22-28° C., accompanied by gentle shaking.

In a further embodiment, the isolated protoplasts are separated from the cell debris by conventional methods selected from flotation method using sucrose density gradient.

In yet another embodiment, the purified protoplasts are fused using fusion agent such as high pH/Ca⁺⁺.

In another embodiment, the fusogen treated protoplasts are allowed to develop into microcolonies on Kao's medium containing 0.5M glucose, 1.0 mg/l 2,4-D, 1.0 mg/l kinetin, at 22-28° C. and low light intensity.

In an embodiment, , the hybrid microcolonies are selected from the milieu of growing microcolonies using appropriate selection agents such as antibiotics selected from hygromycin and phosphinothricin.

In still another embodiment, the selected microcolonies are allowed to develop into calli on K3 medium comprising 1.0 mg/l 2,4-D, 1.0 mg/l kinetin, 20 mg/l hygromycin, 10 mg/l phosphinothricin and incubated at 22-28° C., in10-14 h day-night cycle.

In another embodiment, the calli are transferred to Murashige and Skoog (MS) medium containing 10 mg/lBAP, 1.0 mg/l NAA, 20 μM AgNO₃, 20 mg/l hygromycin, 10 mg/l phosphinothricin and incubated at 22-28° C., 10-14 h day-night cycle for shoot regeneration

In an embodiment, the differentiated shoots transferred to MS medium comprising 2.0 mg/l IBA at 22-28° C., in 10-14 h day-night cycle for development of roots.

In another embodiment, the rooted plantlets obtained in step 7 (viii) are transferred to soil for further development into plants.

In yet another embodiment, the hybrid nature of the plants is determined by RAPD analysis.

In a further embodiment, the organelle composition of the plants is analyzed using RFLP method.

In a further embodiment, the chloroplast type of the hybrid plants is determined by Southern hybridization using heterologous chloroplast gene probes.

In an embodiment, the mitochondria type of the hybrid plants is ascertained through Southern hybridization using heterologous mitochondrial gene probes and cosmid clones.

In still another embodiment, the method for the development of hexaploid cytoplasmic male sterile ‘oxy’ CMS Brassica plants further comprises back-crossing the hexaploid ‘oxy’ CMS Brassica somatic hybrid plants with Brassica napus plants to produce F1 generation.

In yet another embodiment, the B. napus is B. napus var. Indian Synthetic Napus 706.

In still another embodiment, in the method for the development of hexaploid ‘oxy’ CMS Brassica plants, F1 is subjected to back-crossing to B. napus to produce BC1.

In yet another embodiment, in the method for the development of hexaploid ‘oxy’ CMS Brassica plants, BC1 is subjected to back-crossing to B. napus to produce BC2.

In a further embodiment, the BC2 plant is subjected to back-crossing to B. napus to produce BC3

In an embodiment, the BC3 plant is subjected to back-crossing to B. napus to produce BC4.

In yet another embodiment, BC4 is subjected to back-crossing to B. napus to produce BC5. In still another embodiment, the improved ‘oxy’ CMS BC5 B.napus plants are subjected to molecular analysis of chloroplast DNA to establish the faithful transmission of the ‘oleracea’ type chloroplasts from hexaploid ‘oxy’ CMS Brassica plants as defined hereinabove to the improved ‘oxy’ CMS BC5 B. napus plants.

The invention is illustrated with reference to the following accompanying drawings and examples.

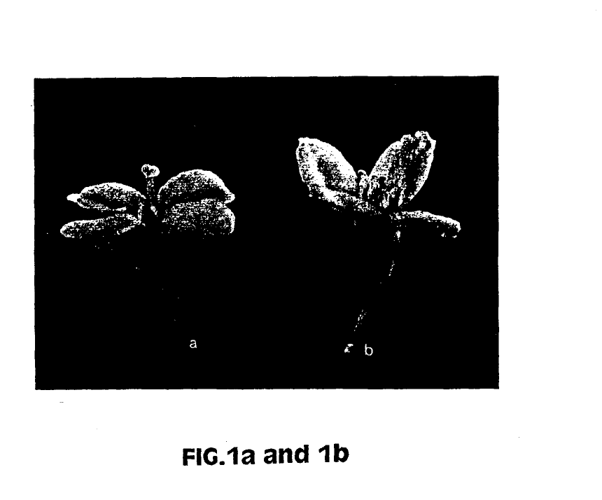

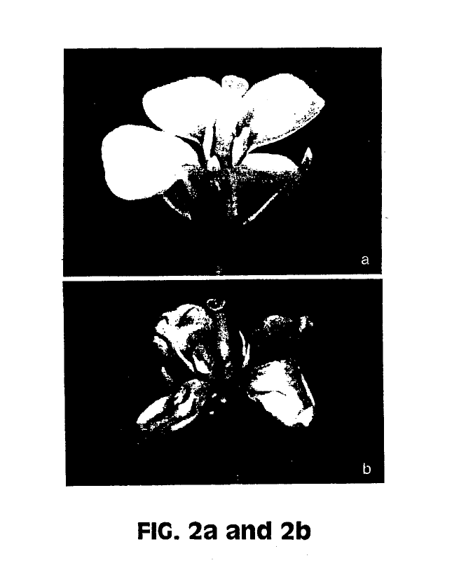

FIGS. 1a,b: are photographs depicting flowers of (a) ‘oxy’ CMS Brassica juncea (AABB) and (b) normal B. oleracea (CC) p FIGS. 2a,b: are photographs depicting two types of variations in the floral morphology of the sterile hexaploid AABBCC Brassica somatic hybrid plants; (a) flower with normal petals and rudimentary anthers, (b) flower with crinckled petals and feathery anthers

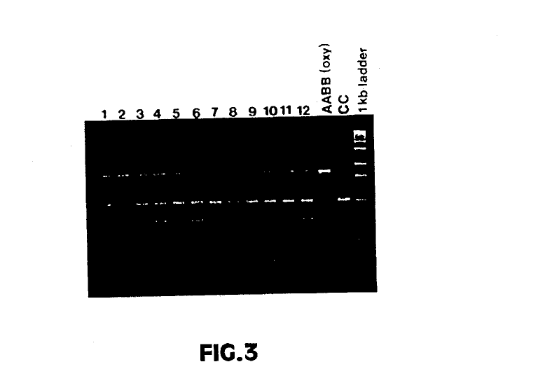

FIG. 3 is a photograph depicting the gel electrophoretic pattern that represents the RAPD profile of total DNA isolated from 12 hexaploid (AABBCC) ‘oxy’ CMS Brassica somatic hybrid plants (lane 1-12) and their parents ‘oxy’ CMS Brassica juncea (hm⁺, AABB, lane 13) and B. oleracea (ppt⁺, CC, lane 14), generated using primer OPB 10. Lane on the extreme right represents the standard DNA fragment size markers.

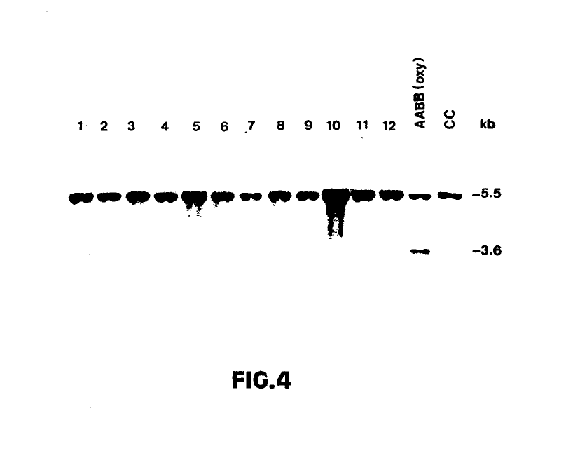

FIG. 4: is a photograph depicting the Southern hybridization pattern representing the RFLP profile of Eco RI digested total DNA isolated from 12 hexaploid (AABBCC) ‘oxy’ CMS Brassica somatic hybrid plants (lane 1-12) and their parents ‘oxy’ CMS Brassica juncea (hm⁺, AABB, lane 13) and B. oleracea (ppt⁺, CC, lane14) that have been probed with a heterologous chloroplast gene probe psbD. The numbers along the right margin of FIG. 2 represent a non-linear scale, in kilobase pairs (kb), of DNA fragment sizes

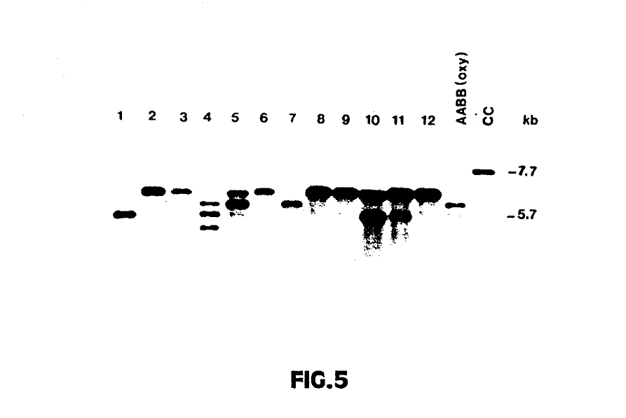

FIG. 5: is a photograph depicting the Southern hybridization pattern representing the RFLP profile of Hind III digested total DNA isolated from 12 hexaploid (AABBCC) ‘oxy’ CMS Brassica somatic hybrid plants (lane 1-12) and their parents ‘oxy’ CMS Brassica juncea (hm⁺, AABB, lane 13) and B. oleracea (ppt⁺, CC, lane14) that have been probed with a mitochondrial gene probe atp A. The numbers along the right margin of FIG. 4 represent a non-linear scale, in kilobase pairs (kb), of DNA fragment sizes.

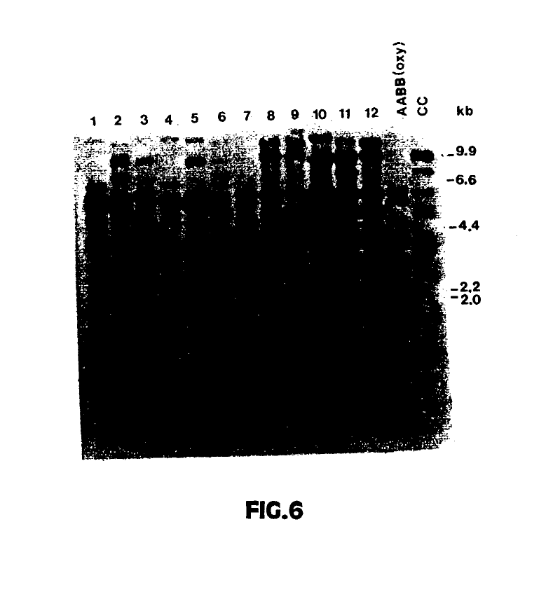

FIG. 6: is a photograph depicting the Southern hybridization pattern representing the RFLP profile of Hind III digested total DNA isolated from 12 hexaploid (AABBCC) ‘oxy’ CMS Brassica somatic hybrid plants (lane 1-12) and their parents ‘oxy’ CMS Brassica juncea (hm⁺, AABB, lane 13) and B. oleracea (ppt⁺, CC, lane14) that have been probed with a cosmid clone pCos 42 derived from B. oxyrrinha mitochondrial DNA. The numbers along the right margin of FIG. 4 represent a non-linear scale, in kilobase pairs (kb), of DNA fragment sizes.

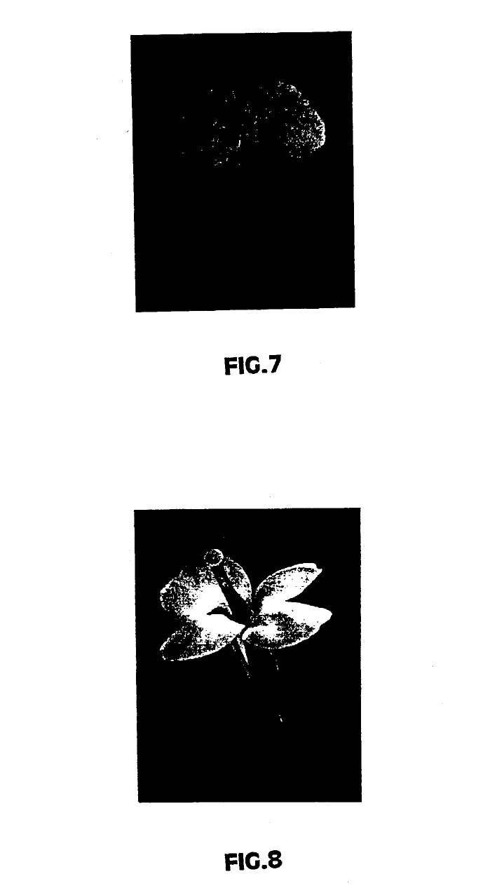

FIG. 7: is a photograph depicting a normal (AACC) var.ISN 706 flower

FIG. 8: is a photograph depicting a flower from BC5 B. napus var. ISN 706 obtained through backcrossing with hexaploid male sterile AABBCC Brassica somatic hybrid plant.

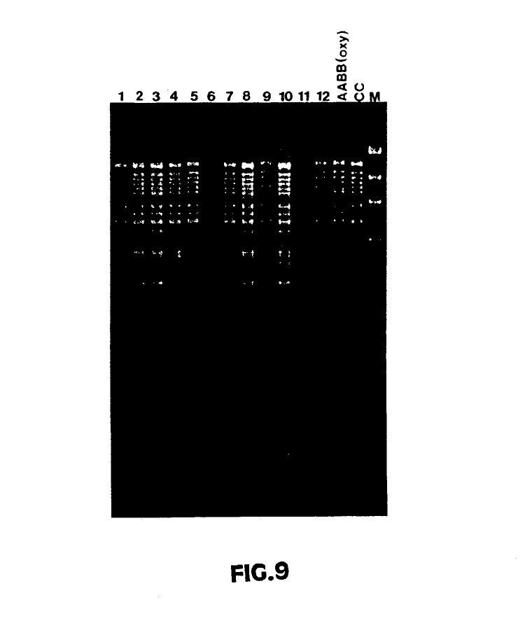

FIG. 9: is a photograph depicting the gel electrophoretic pattern of Hind III digested chloroplast DNA isolated from 12 ‘oxy’ CMS B. napus lines (lane 1-12) at BC5 generation and ‘oxy’ CMS Brassica juncea (hm⁺, AABB, lane 13) and B. oleracea (ppt⁺, CC, lane14). Lane on the extreme right represents the standard DNA fragment size markers.

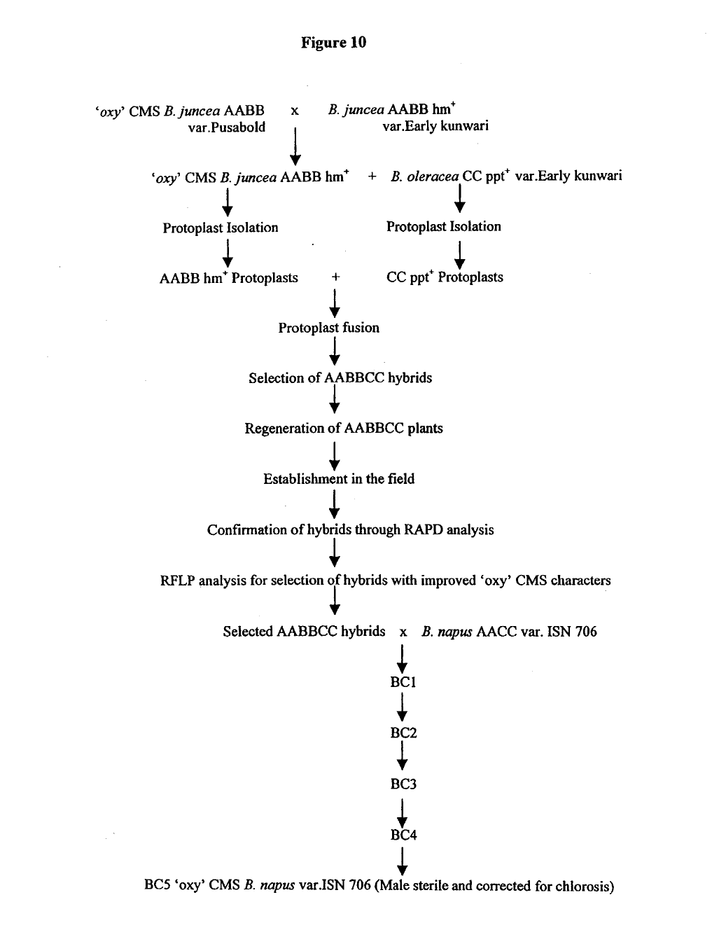

FIG. 10: is a schematic flow diagram describing the methodology of generating the improved hexaploid ‘oxy’ CMS Brassica somatic hybrid plants and further transferring these improved cytoplasmic combinations to tetraploid Brassica napus plants. It comprises the steps of development of the fusion parents containing antibiotic resistance markers followed by isolation of protoplasts from these parents, fusion of the protoplasts using high pH/Ca⁺⁺ method and regeneration of the selected hybrids, screening of the hybrids at molecular level for nuclear and organelle compositions and back-cross breeding of the selected hybrids to tetraploid Brassica napus var.ISN 706 for five generations.

The invention is described in greater detail hereinafter, with reference to the accompanying drawings and examples, which are provided as mere illustrations of the invention and should not be construed to limit the scope thereof in any manner.

Examples of the Brassica plant usable in the present method include any plant to which CMS of Brassica oxyrrhina plant has been introduced. In the present study, Indian mustard B. juncea (AABB), having CMS of ‘oxyrrhina’ has been used as one of the parent plants for generating AABBCC somatic hybrids.

Examples of the B. oleracea (CC) plant usable in the present method include any plant line or cultivar. In the present invention, B. oleracea (var. Early Kunwari) has been preferably employed as the second parent plant for generating the somatic hybrids.

According to the present invention, the problem of chlorosis, associated with ‘oxy’ CMS can be rectified by means of somatic cell hybridization. In case of somatic cell hybridization, the protoplasts derived from Brassica juncea (AABB) plants and B. oleracea (CC) plants mentioned above are first prepared and then fused together. The preparation of the protoplasts can be carried out according to any of the standard procedures. Any part of a seedling (hypocotyls, cotyledons, roots) or a supple young plantlet (leaves, internodes, petioles, etc) is finely chopped in an isotonic solution containing cell wall digesting enzymes such as cellulase and macerozyme and incubated at 22-28° C. overnight (14-17 hours) in dark. After incubation, the slightly plasmolyzed protoplasts are released from the confines of the cell wall by gently agitating the enzyme treated cells in the incubation solution. Next, the protoplasts are separated from the cellular debris by floatation method using a sucrose density gradient, where the heavier cellular debris are pelleted down allowing the lighter protoplasts to float as a band at the interface of the two solutions having different densities.

The fusion of purified protoplasts can be accomplished in several ways, e.g. by using polyethylene glycol, electric field induced fusion or high pH/Ca⁺⁺ treatment. The present invention relates to the use of high pH/Ca⁺⁺ treatment for effecting protoplast fusion. Fusion is carried out following the method described by Keller and Melchers (1973 Z Naturforsch 28: 737-741) with some modifications. Protoplasts of B. juncea and B. oleracea are suspended in 1:1 ratio, at a total density of 1 to 2×10⁶ cells/ml, in CPW solution (Frearson et al., 1973 Dev Biol 33:130-137; Table1) containing 9% mannitol and 3% sucrose (CPW 9M). Fusogen (0.05 M Glycine-NaOH buffer, 1.1% CaCl₂, 9% mannitol, pH 10.4) is gently added to the protoplast mixture and subsequently incubated at 45° C. for 10 min

TABLE 1
Composition of the CPW Salt Solution* (Frearson et al 1973)

| Component | Concentration (mg/l) |
|---|---|
| KH₂PO₄ | 27.2 |
| KNO₃ | 101.0 |
| CaCl₂.2H₂O | 1480.0 |
| MgSO₄.7H₂O | 246.0 |
| KI | 0.16 |
| CuSO₄.5H₂O | 0.025 |

*pH of the medium is adjusted to 5.8 prior to autoclaving

After incubation, the protoplasts are washed in CPW 9M containing 0.74% CaCl₂ and then suspended at a density of 4×10⁴/ml in Kao's basal medium (Glimelius, 1984 Physiol Plant 61:38-44; Table 2),containing 0.5M glucose, 1.0 mg/l 2,4-D and 1.0 mg/l kinetin. The cultures are incubated at 22-28° C. either in dark or at very low light intensity. The protoplasts divide within 24-72 hours of incubation and form multicellular clusters within 8-10 days. After 10 days the glucose in the above medium is gradually diluted by adding 5-7 ml of 0.1M sucrose thrice at three days interval. After another 3-4 weeks of incubation, numerous pale green, amorphous microcolonies having 0.5 to 1.0 mm diameter are formed.

TABLE 2
Composition of Kao's basal medium* (Glimelius 1984)

| | Concentration (mg/l) |
|---|---|
| Nutrients (Macro) | |
| NH₄ NO₃ | 600 |
| KNO₃ | 1900 |
| CaCl₂.2H₂O | 600 |
| MgSO₄.7H₂O | 300 |
| KH₂PO₄ | 170 |
| KCl | 300 |
| Nutrients (Iron) | |
| FeSO₄ 7H₂O | 27.85 |
| Na₂ EDTA | 37.25 |
| Nutrients (B5 Minor) | |
| KI | 0.75 |
| H₃BO₃ | 3.0 |
| MnSO₄.4H₂O | 10.0 |
| ZnSO₄.7H₂O | 2.0 |
| NaMoO₄.2H₂O | 0.25 |
| CuSO₄.5H₂O | 0.025 |
| CoCl₂.6H₂O | 0.025 |
| Nutrients (Organic) | |
| Na pyruvate | 20.0 |
| Citric acid | 40.0 |
| Malic acid | 40.0 |
| Fumari cacid | 40.0 |
| Fructose | 250.0 |
| Ribose | 250.0 |
| Xylose | 250.0 |
| Mannose | 250.0 |
| Rhamnose | 250.0 |
| Cellobiose | 250.0 |
| Nutrients (B5 Organic) | |
| Inositol | 100 |
| Nicotinamide | 1.0 |
| Pyridoxine HCl | 1.0 |
| Thiamine HCl | 10.0 |

*Medium is filter sterilized, pH of the medium is set at 5.8 prior to sterilization

Selection of the hybrid/fused colonies from the milieu of fused and non-fused parental ones can be achieved in several ways, both at the protoplast level and, after the formation of microcolonies. In the present invention, selection of hybrid products is done after the formation of microcolonies, employing antibiotics as selectable markers. Firstly, genes conferring resistance to different antibiotics are introduced into the fusion partners, thus enabling these plants to grow in the presence of those antibiotics such as hygromycin, kanamycin, phosphinothricin, etc. In the present invention one of the parents ‘oxy’ CMS B. juncea (AABB) has gene conferring resistance to hygromycin (hm⁺) while the other parent, normal B. oleracea (CC) has gene conferring resistance to phosphinothricin (ppt⁺). Thus in the present invention, hygromycin and phosphinothricin are used as the selectable markers for the hybrid colonies.

For the selection of hybrid colonies, microcolonies obtained on Kao's medium are transferred to a callus proliferation medium such as K3 medium (Nagy and Maliga 1976 Z Pflanzenphysiol 78:453-455; Table 3), having 10 mg/l 2,4-D, 1.0 mg/l kinetin, 20 mg/l hygromycin and 10 mg/l phosphinothricin and incubated at 22-28° C., 14-16 h photoperiod.

TABLE 3
Composition of the K3 medium* (Nagy and Maliga, 1976)

| | Concentration (mg/l) |
|---|---|
| Nutrients (Macro) | |
| KNO₃ | 2500 |
| NH₄HO₃ | 250 |
| MgSO₄.7H₂O | 250 |
| CaCl₂.2 H₂O | 900 |
| NaH₂PO₄ | 150 |
| (NH₄)₂ SO₄ | 134 |
| Nutrients (Iron) | |
| FeSO₄ 7H₂O | 27.85 |
| Na₂ EDTA | 37.25 |
| Nutrients (B₅ Minor) | |
| KI | 0.75 |
| H₃BO₃ | 3.0 |
| MnSO₄.4H₂O | 10.0 |
| ZnSO₄.7H₂O | 2.0 |
| NaMoO₄.2H₂O | 0.25 |
| CuSO₄.5H₂O | 0.025 |
| CoCl₂.6H₂O | 0.025 |
| Nutrients (organic) | |
| Thiamine | 10 |
| Pyridoxine | 1 |
| Niacin | 1 |
| Inositol | 100 |

*pH of the medium is adjusted to 5.8 prior to autoclaving

The colonies that continue to grow on this medium, are subsequently transferred to shoot induction medium such as Murashige and Skoog (MS; 1962 Physiol Plant 15:472-493: Table 4) containing 1.0 mg/l BAP, 1.0 mg/l NAA, 20 μM AgNO3, 20 mg/l hygromycin, and 10 mg/l phosphinothricin. A total of 591 rapidly growing colonies are transferred for shoot regeneration. Approximately, 5% of these colonies differentiated shoots. In the present invention the regenerated shoots are further transferred to a suitable medium such as, MS solid medium containing 2 mg/l IBA, 20 mg/l hygromycin, and 10 mg/l phosphinothricin, for the development of roots. Finally the rooted plantlets, when about 2-4 cm tall, are transferred to soil under containment during the growing season and grown to maturity to obtain Brassica somatic hybrid plants to which ‘oxy’ CMS had been introduced.

TABLE 4
Composition of Murashige and Skoog (1962) medium*

| | Concentration (mg/l) |
|---|---|
| Nutrients (Macro) | |
| KNO₃ | 1900 |
| CaCl₂.2H₂O | 440 |
| MgSO₄.7H₂O | 370 |
| NH₂NO₃ | 1650 |
| KH₂PO₄ | 170 |
| Nutrients (Micro) | |
| KI | 0.83 |
| CoCl₂.6H₂O | 0.025 |
| H₃BO₃ | 6.2 |
| Na₂MoO₄.2H₂O | 0.25 |
| MnSO₄.4H₂O | 22.3 |
| CuSO₄.5H₂O | 0.025 |
| ZnSO₄.7H₂O | 8.6 |
| Glycine | 2.0 |
| Nutrients (Organic) | |
| Inositol | 100 |
| Nicotinic Acid | 0.5 |
| Pyridoxine HCl | 0.5 |
| Thiamine HCl | 0.1 |
| Nutrients (Iron) | |
| FeSO₄.7H₂O | 27.85 |
| Na₂EDTA | 37.25 |
| Sucrose | 30.0 (g/l) |

*pH of the medium is adjusted to 5.8 prior to autoclaving

A total of 121 somatic hybrid plants that are transferred to the soil under containment, show vigorous (with a few exceptions) growth and attained an average height of 1.3 m within 45-60 days of transfer to soil. The plants have oblong to lanceolate leaves with serrated or entire margins and smooth, non-hirsute surface, 3-6 primary branches, and 6-10 secondary branches. Each of these branch bear terminal and/or axillary, indeterminate racemose inflorescence. Flowering is initiated after 60-70 days. Flower morphology varied from normal to different degrees of abnormality, such as presence or absence of petals, scale like or crinkled petals, presence or absence of nectaries, position, number (2-6) and size of stamens, size and shape of anthers, straight or curved stigma, etc. Both the normal and abnormal type flowers are distinguished as pollen producing and non-producing types. The plants are screened for pollen fertility through morphological observations, such as FDA (Fluorescein diacetate) test and by covering the inflorescence by butter paper bags (selfing) and scoring for pod and seed set after the required length of time. Plants that set seed under the bag are rejected for being fertile and those that did not set seed are retained for being sterile plants. Only one plant showed the presence of 25% viable pollen grains by FDA test. However, even this plant did not produce any seed on selfing.

The hybrid nature of these plants is confirmed by molecular means such as RAPD analysis following Mukhopadhyay et al. (1994 Theor Appl Genet 89:19-25) using random primers from Operon Technologies (USA) (FIG. 1). Fifty-two sterile hybrid plants are then selected randomly and subjected to molecular analysis for mitochondria and chloroplast genomes following standard protocols. Total DNA is isolated from expanded leaves of the selected somatic hybrids and parent plants following Dellaporta et al., 1983 (Plant Mol Biol Rep 1:19-21). For chloroplast genome analysis DNA is digested with restriction enzyme Eco RI, electrophoresed and transferred to nylon membranes and hybridized with two chloroplast specific heterologous probes. Out of the 52 hybrids, 30 show ‘oleracea’ and 22 show ‘oxy’ type chloroplasts. (FIG. 2). None of the hybrids exhibit either the presence of a mixture of parental chloroplasts or chloroplast recombination. For mitochondrial genome analysis the total DNA is digested with Eco RI or HindIII and hybridized to eleven heterologous gene probes and eight overlapping cosmid clones of B. oxyrrhina mitochondrial DNA. Extensive rearrangements in the mitochondrial genome are observed (FIG. 3). The rearrangements are either in the form of appearance of novel bands, a combination of novel and parental bands, or a combination of parental bands. Mitochondrial genomic regions representing four probes namely, cox I, cox 2, atp 6, and atp A were found to show the most frequent variations in the RFLP patterns

Twenty-nine of the 52 morphologically sterile hexaploid hybrid plants that are analyzed for organelle composition show the presence of both ‘oleracea’ type chloroplast and recombined CMS ‘oxy’ mitochondrial genome. These plants are then back-crossed to tetraploid B. napus var. ISN 706, using conventional breeding methods to facilitate simultaneous transfer of the desirable cytoplasmic combinations. Pollen are collected from the pollen donor parent B. napus var. ISN 706 and used for pollinating the selected AABBCC plants. The art of such breeding techniques are known to the skilled breeders. Three to five inflorescence are selected from each plant and 6-12 buds from each inflorescence (depending on the availability) are pollinated and covered with butter-paper bags. Nineteen out of the twenty-nine hybrids set seed following pollination. Siliqua development is normal and the pods are 4-5 cm long. The number of seeds/pod is around 4-6/pod; less compared to that in a normal B. napus (approx. 22-24/pod).

Upon maturity, seeds are harvested from individual plants and planted in the next growing season under containment condition. Seeds obtained from three of the hybrid plants are shriveled and failed to germinate. Seeds from rest of the 16 plants, sown in 16 separate lines, germinate within 5-6 days and the BC1 plants grow normally. Each line contained 10 plants. The plants show green, oval to oblong, smooth non-hirsute leaves, have 4-5 primary and 6-8 secondary branches bearing indeterminate, racemose type inflorescence and are about160 cm tall. The plants flower within 80-90 days Some of the AABBCC plants that show petal-less flowers, in BC1 generation develop either crinckled or normal petals varying from 1-4 in number. Whereas in general, the flowers have rudimentary or empty anther sacs, pollen development is noted in some plants in BC1 generation. However, on selfing these plants fail to set seed, confirming their sterile nature. Two phenotypically best plants are selected from each of the 16 lines and these selected BC1 plants are back-crossed to B. napus var. ISN 706 (pollen parent) to produce BC2 seeds following protocol described as above. Siliqua develop in situ and upon maturity the seeds are harvested from individual plants and seeds from two plants belonging to the same line are bulked. All the plants produce seeds having on an average 6-10 seed/pod. The pod length vary between 4-5 cm.

The BC2 seeds are planted as 16 separate lines in the next growing season under containment conditions. Seeds from all the plants germinate within 5-6 days to produce BC2 plants. These plants have smooth, green, oblong leaves with slight leathery texture, 4-5 primary and 6-8 secondary branches bearing indeterminate, racemose type inflorescence. The plants flower within 80-90 days. The normalization of petal morphology continues in BC2 generation as more number of previously petal-less plants showed petal development. All the BC2 plants have morphologically male sterile flowers, i.e. flowers with either rudimentary or empty pollen sacs. However, as in BC1, some BC2 plants show pollen formation but fail to form seed on selfing, confirming their male sterile nature. Two of the best BC2 plants are selected from each line and back-crossed to B. napus var. ISN 706 (pollen parent). The pods are 5-6 cm long, seed set is normal, and each pod developed about 12-15 seeds. Upon maturity the BC3 seeds are harvested from individual plants and seeds from two plants belonging to the same line are bulked.

The BC3 seeds harvested from 16 different lines are sown in 16 different rows under containment condition in the next growing season. The seeds germinate and BC3 plants grow normally. These plants have smooth, green, oblong leaves with leathery texture, 5-6 primary and 6-8 secondary branches bearing indeterminate, racemose type inflorescence and thus largely resembled the B.napus pollen parent. The plants flower within 90 days. Most of the plants had flowers with normal petal, and rudimentary anthers. Some flowers that show the presence of pollen grains fail to produce seed on selfing thus confirming their sterile nature. Two of the best BC3 plants are selected from each line and backcrossed to B. napus var. ISN 706 (pollen parent). The pods are 6-7 cm long, seed set is normal, and each pod contains about 18-20 seeds. Upon maturity the BC4 seeds are harvested from individual plants and seeds from two plants belonging to the same line are bulked.

The BC4 seeds are sown in the next growing season maintaining the 16 lines separately. The seeds germinate and BC4 plants develop normally. These plants, by and large, resemble their pollen parent morphologically, i.e. the plant height, leaf and flower morphology, number of primary and secondary branches, days to flowering, pod length, number of seed/pod, etc. The leaves of the BC4 plants appear green, smooth, leathery, and oblong. The plants bear 5-6 primary and 3-4 secondary branches and flower within 90-95 days of planting. The BC4 plants are about 170 cm. Upon pollination with B. napus var. ISN 706 develop 6-7 cm long pods bearing 20-24 seeds/pod. The average pod density on the main shoot is observed to be 0.9/cm. The male sterile nature of all the BC4 plants are established by selfing inflorescence on each plant, irrespective of presence or absence of pollen on the flowers. None of the plants produce seeds on selfing thus confirming their male sterile nature. Upon maturity the BC5 seeds are harvested from individual plants and seeds from two plants belonging to the same line are bulked.

The recurring pollen parent Brassica napus var ISN 706 sown under normal growing conditions shows the following morphological characteristics: the plants on an average attain a height of about 170 cm while the extremes of height may vary between156 cm to 185 cm. Similarly, the numbers of primary and secondary branches vary between 4 to6 (avg.5) and 2 to4 (avg.3), respectively. The length of main shoot is usually around 85 cm. However, some plants may have main shoot as long as 60 cm only. Brassica napus usually comes to flowering within 90-92 days of planting. The number of pods on the main shoot may vary between 59 to 78, the average being 69. Pod density on the main shoot ranges from 0.81 to 1.09/cm and the number of seeds/pod averages around 22.

The BC5 plants are grown similarly as in BC4 generation. These plants resemble the pollen parent B. napus var. ISN 706 and all the plants are male sterile. The molecular composition of the BC5 plants, specially with reference to chloroplasts, is analyzed to confirm the fidelity of the transmission of the cytoplasmic characters through the five back-cross generations. Chloroplasts DNA is isolated from the leaves of BC5 plants and the parents following Kemble 1987 (Theor Appl Genet 73: 364-370) and digested with Eco RI and HindIII. Molecular analysis confirmed the presence of ‘oleracea’ type chloroplast (FIG. 4) thus confirming the transmission of improved ‘oxy’ CMS character from AABBCC hybrids to the BC5 plants. Thus in the present invention B. napus plants containing improved ‘oxy’ CMS cytoplasm are developed.

### EXAMPLES

### Example 1

Plant Material:

Brassica juncea (AABB) var. ‘Pusa bold’ which carries ‘oxy’ CMS cytoplasm was crossed sexually to hygromycin resistant (Hm⁺) B. juncea var. ‘RLM198’ (Pental et al., 1993 Plant Cell Rep. 12: 462-467). The F1 seeds thus obtained were germinated in vitro and the seedlings screened for resistance to hygromycin by re-rooting on hygromycin containing medium. The resistant seedlings were multiplied and maintained in vitro and used in this invention for further experiments.

Seeds of Brassica oleracea (CC) variety ‘Early kunwari’ carrying phosphinothricin resistance (ppt⁺, Mukhopadhyay et al., 1991 Plant Cell Rep.10:375-379) were also used. The seeds were germinated and seedlings screened for resistance to phosphinothricin by re-rooting the seedlings on phosphinothricin containing medium in vitro. The resistant seedlings were multiplied and maintained in vitro and used in this invention for further experiments.

### Example 2

Seed Sterilization and Germination:

The seeds of B. oleracea and F1 ‘oxy’ CMS B. juncea were dipped for 10 min in 0.05% mercuric chloride for surface sterilization. Then the seeds were rinsed thoroughly with sterile distilled water and placed on MS solid basal medium (Murashige and Skoog, 1962 Physiol Plant 15:472-493, see Appendix 1a for detailed composition) with 3% sucrose for germination. The germinated seedlings of ‘oxy’ CMS B. juncea were screened for resistance to hygromycin (hm⁺) by re-rooting on R1 medium (MS medium with 2.0 mg/l IBA, and 20 mg/l hygromycin). The hm⁺ shoots of ‘oxy’ CMS B. juncea thus obtained were multiplied and maintained on this medium. The germinated seedlings of B. oleracea were similarly screened for resistance to phosphinothricin by re-rooting on R2 medium (MS medium containing 1.0 mg/l IBA, and 10 mg/l phosphinothricin). The resistant shoots (ppt⁺) were multiplied and maintained on shoot multiplication medium (MS containing 0.005 mg/INAA, 0.05 mg/l kinetin, 50 mg/l casein hydrolysate). All the shoot cultures were maintained at 22-28° C. and 16 h photoperiod.

### Example 3

Isolation of Protoplasts:

Shoots of hm⁺ ‘oxy’ CMS B. juncea and ppt⁺⁺ B. oleracea were freshly subcultured on R1 and R2 (as mentioned in example 2), respectively, and grown for 15 days prior to the isolation of protoplasts. Petioles and the soft, supple green internodes were used for the isolation of protoplasts. Petioles and internodes as mentioned, were explanted and immersed in an isotonic CPW (Frearson et al., 1973 Dev Biol 33:130-137; see Appendix 1b for detailed composition) solution contianing 9% mannitol (CPW 9M), cell wall digesting enzymes (1% cellulase R-10 and 0.5% macerozyme R-10) and put for overnight (14-16 h) incubation in dark at 22-25° C. with slow shaking (50 rpm). The plasmolyzed protoplasts were gently squeezed and sieved through 0.16 mm mesh, washed twice in CPW 9M by centrifuging at 580 g for 10 min. and re-suspended in 2 ml of CPW 9M. Protoplast suspension was gently over-layered with CPW 18 S (18% sucrose) and centrifuged at 400 g for 10 min. Purified protoplasts gathered as a band at the inter-phase were harvested and washed twice in CPW 9M.

### Example 4

Fusion of Protoplasts:

Protoplast fusion was carried out by a high pH/Ca⁺⁺ treatment. A 1:1 mixture of ‘oxy’ CMS B. juncea (AABB, hm⁺) and B. oleracea (CC, ppt⁺) were suspended in 0.5 ml CPW 9M containing 3% sucrose, 1.0 mg/l BAP, 1.0 mg/l NAA. Four milliliters of fusogen (0.05 M glycine-NaOH buffer, 1.1% Ca Cl₂.6H₂O and 9% mannitol, pH 10.4) was gently added to the protoplast mixture, which was subsequently incubated at 45° C. for 10 min. Following incubation, the protoplasts were pelleted by centrifugation at 500 g for 5 min. and re-suspended and washed in CPW 9M with 0.74% CaCl₂.6H₂O.

### Example 5

Culture and Selection of the Fusion Products:

The fusogen treated protoplasts according to example 4 were plated at a density of 4-5×10⁴/ml in MSP medium [Kao's basal medium (as mentioned by Glimelieus, 1984 Physiol Plant 61: 38-44; see Appendix 1c for detailed composition) with 0.5M glucose, 1.0 mg/l BAP, 1.0 mg/l NAA]. The cultures were kept at 22-25° C. in dark. After 10 days of culture the medium was diluted thrice at three-day intervals with MSP medium modified by only replacing 0.5M glucose with 0.1M sucrose. After 4 weeks of culture 0.5-1.0 mm large microcolonies appeared. These microcolonies were over-layered on selection medium i.e.K3 basal medium (Nagy and Maliga,1976 Z. Pflanzenphysiol. 78: 453-455; see Appendix 1d for detailed composition) containing 0.5M glucose, 1.0 mg/l BAP, 1.0 mg/l NAA, 20 μMAgNO₃, 20 mg/l hygromycin,10 mg/l phosphinothricin, 0.25% agarose] and incubated at 22-24° C. with 16 h photoperiod for the selection of hybrid colonies.

### Example 6

Plant Regeneration:

The hybrid colonies of example 5 that continued to grow on the selection medium were transferred to the shoot regeneration medium i.e. MS basal medium containing 1.0 mg/l BAP, 1.0 mg/l NAA, 20 μMAgNO₃, 20 mg/l hygromycin and 10 g/l phosphinothricin. The shoots that regenerated on this medium were subsequently rooted on MS basal medium with 2 mg/l IBA, containing 20 mg/l hygromycin and 10 mg/l phosphinothricin.

### Example 7

Molecular Analysis of the Regenerated Plantlets Obtained Through Protoplast Fusion

a) Isolation of Total DNA:

Total DNA was isolated from the fully expanded leaves of the regenerated plantlets growing in the field under containment and the parents following Dellaporta et al., 1983 (Plant Mol Biol Rep 1: 19-21). One gram of leaf tissue was powered finely in liquid nitrogen and homogenized in 15 ml extraction buffer containing 100 mM tris-HCl, pH 8; 50 mM EDTA, pH 8; 50 mM NaCl and 10 mM β-mercaptoethanol. Next 1 ml of 20% SDS was added to the homogenate, mixed thoroughly and incubated at 65° C. for 10 min. Five ml of 5M potassium acetate was added to the mixture and again incubated at 0° C. for 20 min. The content was then centrifuged at 25000 g for 20 min., and the supernatant filtered through miracloth. DNA was precipitated by adding 10 ml of iso-propanol to the filtered supernatant and centrifuging it at 20000 g for 10 min. The pellet was dissolved in 0.5 ml of TE buffer (50 mM Tris HCl, pH 8.0; 10 mM EDTA, pH 8.0). DNA was re-precipitated by adding iso-propanol and finally dissolved in TE buffer (10 mM Tris HCl, pH 8.0; 1 mM EDTA, pH 8.0), and further purified on cesium chloride (CsCl). For every 1 ml of DNA suspension 0.95 g of solid CsCl was added and allowed to dissolve completely. Ethidium-bromide was added to a final concentration of 400 μg/ml and then the DNA suspension was centrifuged overnight at 55000 rpm in Beckman ultracentrifuge. Following centrifugation Ethidium-bromide and CsCl were removed and DNA was precipitated by adding two volumes of ethanol and re-dissolved in TE buffer.

b) Nuclear DNA Composition:

Characterization of the nuclear composition of the hexaploid regenerated plantlets was effected by RAPD analysis using two 10-mer primers, OPB 8 and OPB10 (Operon Technologies, USA) and subjecting to PCR amplification of the DNA following Mukhopadhyay et al., 1994 (Theor Appl Genet 89: 19-25). A 25 μl PCR reaction mixture was prepared containing 1×reaction buffer (Perkin Elmer-Ceteus), 150 μM dNTPs, 2 mM MgCl₂, 1u Stoffel fragment (Perkin Elmer-Ceteus), 15 ng primer, and 50 ng of sample DNA. The DNA amplification was done for 45 cycles in Perkin Elmer thermal Cycler 9600 where the denaturation was done at 92° C. for 1 min., annealing at 35° C. for 1 min., and extension at 72° C. for 2 min. except in the first cycle where denaturation was done for 2 min. Amplified products were electrophoresed on 1.8% agarose gel and analyzed by staining with ethidium bromide. Parent specific PCR amplified products were observed in the regenerated plantlets, establishing their hybrid nature (FIG. 1). The experiment was repeated for confirming its reproducibility.

c) Analyzing for Chlorolast DNA Composition:

For analyzing the chloroplast genome, total DNA was digested with Eco RI, electrophoresed and transferred onto nylon membranes (Hybond-N, Amersham, UK) and hybridized to two heterologous probes of chloroplast origin, namely rbc L (Zurawski et al., 1981 Nucleic Acid Res 9: 3251-3270) and psb D (Alt et al., 1984 Current Genet 8: 597-606) following the method of Pradhan et al.,1992 (Theor Appl Genet 85: 331-340)

d) Analyzing for Mitochondrial DNA Composition:

For analyzing mitochondrial DNA, total DNA was digested with Eco RI and Hind III and electrophoresed on 0.8% agarose gel, transferred to nylon membranes and hybridized to 11 mitochondrial gene probes namely, atp A, atp 6, atp 9, cox 1, cox 2, cox 3, cob, rrn5-18, rrn 26, nad 3 and nad 4 and 8 overlapping cosmid clones from B. oxyrrhina mitochondrial DNA covering about 190 kb of the mitochondrial DNA (Arumugam et al., 1996 Theor Appl Genet 92: 762-768).

e) Protocols for Restriction Digestion and Southern Hybridization:

Five μg of total DNA was digested overnight with different restriction endonucleases in a 40 μl reaction volume containing 30 u of restriction enzyme as recommended by the suppliers. The digested DNA was electrophoresed on 0.8% agarose gel, transferred to nylon membranes after treating the gel with denaturation (0.2M NaOH, 6M NaCl) and neutralization (0.5M Tris-HCl, pH 7.5, 1.5M NaCl) solutions.

Southern hybridization was done by treating the membrane for 6 h at 42° C. in prehybridization buffer containing 50% formamide, 0.1% Denhardt's solution, 5×SSC, 5% Dextran sulphate, 1% SDS and 200 ng of Salmon sperm DNA. Following prehybridization the labeled probe was denatured and added to the membrane. The membrane was then hybridized for 16 h at 42° C. Labeled probes were prepared using Amersham multiprime labeling kit following manufacturers instructions. After hybridization the membranes were washed twice in 2×SSc for 15 min. at room temperature and once at 30° C. in 0.2×SSC, 0.1% SDS at 65° C. Subsequently the membranes were exposed overnight to x-ray film (Kodak, X-Omat). The banding patterns were resolved by developing these x-ray films.

f) Isolation and Analysis of Chloroplast DNA from BC5 Plants:

The isolation was carried out at 4° C. Five gram of leaf tissue was homogenized in pestle and mortar with 70 ml buffer A (0.35M sorbitol, 50 mM Tris-HCl pH8.0, 5 mM EDTA, 0.1% BSA, 15 mM β-mercaptoethanol, 1 mM Spermin, and 1 mM Spermidine). The homogenate was filtered through 4 layers of cheese cloth and centrifuged at 1000 g for 10 min. The pellet was suspended in 10 ml of buffer B (0.35M sorbitol, 50 mM Tris-HCl pH8.0, 25 mM EDTA, 15 mM β-mercaptoethanol, 1 mM Spermin, and 1 mM Spermidine), centrifuged at 1000 g for 10 min and the pellet was resuspended in 9.5 ml of buffer B. The suspension was layered onto a step gradient consisting of 0.7 ml 30% sucrose and 18 ml 60% sucrose, both in buffer B and was centrifuged at 25000 rpm for 45 min in a Beckman sw27 rotor. The purified chloroplast collecting at the 30:60 interface was removed with a pipette and diluted with 30 ml buffer B. The content was centrifuged at 1500 g for 15 min. The final pellet was lysed in buffer C (50 mMTris-HCl pH8, 10 mM EDTA, 2% sarkosyl, 0.012% proteinase k) for one hour at 37° C. The cpDNA was extracted after two cycles of phenol-chloroform extraction. The DNA was precipitated overnight at −20° C. after adding two volumes of ethanol. The DNA precipitate was concentrated by centrifugation, washed twice with 70% ethanol and re-suspended in TE buffer.

Two microgram of cpDNA was digested overnight with Eco RI and HindIII following manufacturer's instruction and electrophoresed on 1.0% agarose gel, stained with ethidium bromide and photographed on UV transilluminator.

1. Stable chlorosis-corrected, improved ‘oxy’ CMS Brassica napus (AACC)plants and parts or seeds thereof, whose cytoplasm is provided via protoplast fusion and contains chloroplasts from B. oleracea (CC) and either recombined or native mitochondria from B. oxyrrhina.

2. Improved ‘oxy’ CMS Brassica napus (AACC)plants as claimed in claim 1 wherein, the said plant has the following characteristics:
a) height of 156 cm to 185 cm, average height of about 170 cm,
b) having 4 to 6 primary and 2 to 4 secondary branches, averaging 5 and 3 respectively,
c) main shoot having length of about 85 cm,
d) initiation of flowering within about 90 days,
e) having 59 to 78 pods on the main shoot, average of 69,
f) having pod density of about 0.81 to 1.09 cm on the main shoot,
g) having about 22 seeds/pods, and
h) oblong to lanceolate leaves with slightly serrated or entire margins and smooth, non-hirsute surface.

3. A process of development of stable chlorosis-corrected, improved ‘oxy’ CMS Brassica napus plants as claimed in claim 1, said process comprising the steps of:
i) developing hexaploid cytoplasmic male sterile (CMS) Brassica plants (AABBCC) containing improved ‘oxy’ cytoplasm i.e. chloroplast from Brassica oleracea (CC) and either recombinant or unmodified male sterile mitochondria from Brassica oxyrrhina through the process of somatic cell hybridization,
ii) back-crossing the selected hexaploid male sterile somatic hybrids to B. napus to produce F1 generation,
iii) subjecting the F1 generation to further back-crossings, at least up to BC5 generation to produce stable male sterile lines with improved ‘oxy’ CMS in B. napus, and
iv) verification of the cytoplasmic characteristics of the BC5 B. napus plants by molecular analysis to establish the faithful transmission of the improved cytoplasmic characteristics from the hexaploid somatic hybrids through the five backcross generations.

4. A method as claimed in claim 3 wherein the Brassica napus used for back-crossing in steps (ii) and (iii) is Brassica napus var ISN 706 (Indian Synthetic napus 706).

5. Hexaploid cytoplasmic male sterile ‘oxy’ CMS Brassica plants containing improved ‘oxy’ cytoplasm i.e. chloroplast from Brassica oleracea and either recombinant or unmodified male sterile mitochondria from Brassica oxyrrhina, said hexaploid CMS plants obtained through somatic cell hybridisation.

6. Hexaploid male sterile CMS Brassica plants as claimed in claim 5 wherein the said plant has the following characteristics:
i) height of about 140-190 cm, averaging 165 cm,
ii) oblong to lanceolate leaves with serrated or entire margins and smooth, non-hirsute surface,
iii) 3-6 primary branches, and 6-10 secondary branches bearing terminal and/or axillary, indeterminate racemose inflorescence,
iv) initiation of flowering within 60-70 days,
v) pod length of about 4-5 cm, and
vi) having about 4-6 seeds per pod.

7. A method for the development of hexaploid cytoplasmic male sterile ‘oxy’ CMS Brassica plants containing improved ‘oxy’ cytoplasm as claimed in claim 5, said method comprising the steps of:
(ii) isolation of protoplasts from ‘oxy’ CMS B. juncea plants (AABB) and B. oleracea (cc) plants,
(ii) separation of protoplasts from the cell debris by conventional methods,
(iii) fusion of parental protoplasts using conventional methods,
(iv) cultivation of microcolonies, in a suitable growth media,
(v) selection of hybrid (AABBCC) colonies from the milieu of microcolonies derived from various non-fused parental cells, using appropriate selection agents or markers,
(vi) inducing further growth of the hybrid colonies to form calli and differentiation of shoots from the selected colonies employing suitable media,
(vii) inducing development of roots from the shoots employing a suitable medium,
(viii) transferring the rooted plantlets to soil to obtain ‘oxy’ CMS hexaploid (AABBCC) Brassica somatic hybrid plants,
(ix) screening of the hybrid plants for pollen fertility on the basis of morphological observations,
(x) confirming the hybrid nature of the plants by molecular analysis of their nuclear genomes, and,
(xi) analyzing the organelle compositions for the presence of ‘oleracea’ type chloroplast and recombined ‘oxy’ mitochondrial genome which is indicative of the improved ‘oxy’ CMS systems corrected for chlorosis.

8. A method as claimed in claim 7, wherein the progenitors of hexaploid ‘oxy’ CMS Brassica plants are selected from Brassica juncea (AABB) and B. oleracea (CC) plants.

9. A method as claimed in claim 7 wherein protoplasts are isolated from Brassica juncea and B. oleracea plants.

10. A method as claimed in claim 7 wherein the protoplasts are isolated from plant parts of B. juncea and B. Oleracea selected from finely chopped tissues such as leaves, internodes, and petioles.

11. A method as claimed in claim 7 wherein the tissues of the plant parts are treated with cell wall digesting enzymes selected from cellulase and macerozyme and kept overnight in dark or low light intensity at 22-28° C., accompanied by gentle shaking.

12. A method as claimed in claim 7 where in the isolated protoplasts are separated from the cell debris by conventional methods selected from flotation method using sucrose density gradient.

13. A method as claimed in claim 7 wherein the purified protoplasts are fused using fusion agent such as high pH/Ca⁺⁺.

14. A method as claimed in claim 7 wherein the fusogen treated protoplasts are allowed to develop into microcolonies on Kao's medium containing 0.5M glucose, 1.0 mg/l 2,4-D, 1.0 mg/l kinetin, at 22-28° C. and low light intensity.

15. A method as claimed in claim 7 wherein the hybrid microcolonies are selected from the milieu of growing microcolonies using appropriate selection agents such as antibiotics selected from hygromycin and phosphinothricin.

16. A method as claimed in claim 7 wherein the selected microcolonies are allowed to develop into calli on K3 medium comprising 1.0 mg/l 2,4-D, 1.0 mg/l kinetin, 20 mg/l hygromycin, 10 mg/l phosphinothricin and incubated at 22-28° C., in 10-14 h day-night cycle.

17. A method as claimed in claim 7 wherein the calli are transferred to Murashige and Skoog (MS) medium containing 1.0 mg/lBAP, 1.0 mg/l NAA, 20 μM AgNO₃, 20 mg/1 hygromycin, 10 mg/l phosphinothricin and incubated at 22-28° C., 10-14 h day-night cycle for shoot regeneration.

18. A method as claimed in claim 7 wherein the differentiated shoots transferred to MS medium comprising 2.0 mg/l IBA at 22-28° C., in 10-14 h day-night cycle for development of roots.

19. A method as claimed in claim 7 wherein the rooted plantlets obtained in step 7 (viii) are transferred to soil for further development into plants.

20. A method as claimed in claim 7 wherein the hybrid nature of the plants is determined by RAPD analysis.

21. A method as claimed in claim 7 wherein the organelle composition of the plants is analyzed using RFLP method.

22. A method as claimed in claim 7 wherein chloroplast type of the hybrid plants is determined by Southern hybridization using heterologous chloroplast gene probes.

23. A method as claimed in claim 7 wherein the mitochondria type of the hybrid plants is ascertained through Southern hybridization using heterologous mitochondrial gene probes and cosmid clones.

24. A process as claimed in claim 7 further comprising back-crossing the hexaploid ‘oxy’ CMS Brassica somatic hybrid plants with Brassica napus plants to produce F1 generation.

25. A process as claimed in claim 24 wherein the B. napus is B. napus var. Indian Synthetic Napus 706.

26. A process as claimed in claim 24 wherein the F1 is subjected to back-crossing to B. napus to produce BC1.

27. A process as claimed in claim 26 wherein the BC1 is subjected to back-crossing to B. napus to produce BC2.

28. A process as claimed in claim 27 wherein the BC2 is subjected to back-crossing to B. napus to produce BC3.

29. A process as claimed in claim 28 wherein the BC3 is subjected to back-crossing to B. napus to produce BC4.

30. A process as claimed in claim 29 wherein the BC4 is subjected to back-crossing to B. napus to produce BC5.

31. A process as claimed in claim 30 wherein the improved ‘oxy’ CMS BC5 B.napus plants are subjected to molecular analysis of chloroplast DNA to establish the faithful transmission of the ‘oleracea’ type chloroplasts from hexaploid ‘oxy’ CMS Brassica plants as defined in claim 5 to the improved ‘oxy’ CMS BC5 B. napus plants.
